(19) United States
(12) Patent Application Publication
(10) Pub. No.: US 2023/0364344 A1
(43) Pub. Date: Nov. 16, 2023

(54) LOCKABLE SYRINGE FOR SAFELY DISPENSING A DRUG

(71) Applicant: LABORATOIRE AGUETTANT, LYON (FR)

(72) Inventors: Jeff TONNAR, LYON (FR); Candice MOUTERDE, Le Perreux sur Marne (FR); Astré BOUCHIER, Neuilly sur Seine (FR); Laurent FERRIER, Paris (FR); Loic ARNAUD, Vaugneray (FR)

(73) Assignee: LABORATOIRE AGUETTANT, LYON (FR)

(21) Appl. No.: 18/027,031

(22) PCT Filed: Sep. 17, 2021
(86) PCT No.: PCT/EP2021/075707

§ 371 Date: Mar. 17, 2023

(30) Foreign Application Priority Data
Sep. 18, 2020 (EP) 20306058.7

(51) Int. Cl. A61M 5/315

(52) U.S. Cl. CPC A61M 5/31505; A61M 2205/584; A61M 5/31536

(57) ABSTRACT

A lockable syringe, which includes a syringe body having a longitudinal axis and including a distal end and a proximal end, and a locking mechanism, wherein the locking mechanism is configured to be attached to the proximal end of the syringe body. The locking mechanism includes: a plunger having at least one distal plate, and a rod with visual elements for marking an unlock position; and a locking ring which is free in its rotation about the longitudinal axis of the syringe body, the locking ring has at least two visual indicators on its outer surface, and the locking ring has elements for blocking the plunger stroke in a locked position.

### FIELD OF INVENTION

The present invention relates to the field of medical syringes. Especially, the present invention concerns a lockable syringe for safely dispensing a drug, said lockable syringe having a locking mechanism configured to be attached to the proximal end of the syringe body. The locking mechanism of the invention comprises (i) a plunger having at least one distal plate and a rod with visual means for marking an unlock position; and (ii) a locking ring, free in its rotation, about the longitudinal axis of the syringe body, said locking ring having at least one visual indicator on its outer surface, and said locking ring having means for blocking the plunger stroke in a locked position.

### BACKGROUND OF INVENTION

Syringes suitable for dispensing a drug or a pharmaceutical composition to a subject are particularly interesting for medical applications.

A medical syringe classically comprises a syringe body with a tip, a piston inside the syringe body configured to be in contact with the drug or the pharmaceutical composition pre-filled in said syringe body, and a plunger configured to slidably move the piston inside the syringe body, along the longitudinal axis of said syringe body.

In general, when a practitioner must dispense a drug to a subject from a prefilled syringe, he only has to push the plunger of the syringe for slidably moving the piston inside the syringe body and thus, delivering the drug.

However, it may happen that the practitioner is in a medical environment where several syringes pre-filled with different drugs are at his disposal. In this case, the risk of using the wrong pre-filled syringe is increased.

In order to limit the risk of medical error, there is a need for providing safer pre-filled syringes. Especially, there is a need for making the practitioner more aware of the use of the chosen pre-filled syringe by forcing him to check that the drug to be administered to the patient corresponds to that prefilled in the syringe. Furthermore, the locking mechanism of the pre-filled syringe has to be ergonomic and simple to be used by the operator so that checking step of the drug inside the pre-filled syringe is fast and easy to implement. There is also a need for providing pre-filled lockable syringes by a low-cost and industrializable process.

### SUMMARY

For this purpose, a subject of the invention is a lockable syringe for dispensing a drug, comprising:
a syringe body having a longitudinal axis and comprising a distal end and a proximal end; and
a locking mechanism;

wherein:
said locking mechanism is configured to be attached to the proximal end of the syringe body, and said locking mechanism comprises:
a plunger having at least one distal plate, and a rod with visual means for marking an unlock position; and
a locking ring which is free in its rotation about the longitudinal axis of the syringe body, said locking ring has at least one visual indicator on its outer surface, and said locking ring has means for blocking the plunger stroke in a locked position.

In a conventional manner, within the frame of the invention, the terms “proximal” and “distal” refer to a position of portions of the syringe with reference to an operator (e.g. a dentist or any practitioner), i.e. the term “proximal” refers to a position closer to the operator of the syringe, while the term “distal” refers to a position that is more distant from the operator.

In the lockable syringe of the invention, the dispensing of the drug is achieved by unlocking the locking mechanism of the syringe of the invention. Thanks to the specific arrangement of the locking mechanism, the practitioner is aware of the drug that is about to be dispensed to the patient. In the present invention, the locking system is not suitable to be used when the plunger is pulled backwards (creating negative pressure).

According to one embodiment, the locking mechanism may comprise an adapter configured to be attached to the proximal end of the syringe body and comprising means for receiving the locking ring. Advantageously, the adapter may be a separated molded component or may be molded with the syringe body. In this way, and whatever the molding of the adapter, the locking ring may be easily connected to the proximal end of any kind of syringes. Furthermore, the adapter does not modify the good seal between the syringe body and the plunger.

According to one embodiment, means for receiving the locking ring comprise at least one groove. In this manner, the locking ring is both properly connected to the adapter and is free in its rotation about the longitudinal axis of the syringe body.

According to one embodiment, the adapter is located on the flanges of the syringe body. This specific arrangement allows connecting the locking mechanism on the syringe body while keeping good handling of the device by the practitioner.

According to one embodiment, the locking ring comprises at least two visual indicators, preferably 2, 3, 4, 5, 6, 7 or 8 visual indicators, which may be of the same or different nature. In the lockable syringe of the invention, the presence of at least two visual indicators on the outer surface of the locking ring, forces the practitioner to adjust the position of the locking ring in view of another visual indicator located on the plunger.

The suitable adjustment of at least one of the visual indicators of the locking ring with a visual indicator on the plunger allows unlocking the locking mechanism and finally, the dispensing of the drug pre-filled in the syringe body. Consequently, the risks for dispensing the wrong drug to a subject is strongly reduced because the practitioner needs to align the visual indicator on the rod with the indicator on the locking ring indicating for example the drug present in the syringe. According to one embodiment, the locking ring has a cylindrical shape. According to one embodiment, the shape of the locking ring is not a U-shape nor a C-shape.

According to one embodiment, means for blocking the plunger stroke in a lock position comprise at least one tab. According to one embodiment, when means for blocking the plunger stroke in a lock position comprise at least one tab, the plunger comprises at least one notch on the distal plate configured for receiving said tab and providing an unlock position. In this way, the syringe is initially in a locked configuration in which the plunger cannot be pushed inside the syringe body and consequently, the drug cannot be dispensed. For unlocking the locking mechanism, the operator has to move to a suitable position the tab of the locking ring in front of the notch present on the distal plate of the plunger, said tab and notch being not visible from the operator. In this manner, the operator is made more aware of the action in progress.

According to one embodiment, the notch is marked by a visual indicator on the rod of the plunger. According to one embodiment, the visual indicator of the rod marks only one unlocked position. According to one embodiment, the at least one tab is aligned on one visual indicator present on the outer surface of the locking ring. In this way, the operator has to rotate the locking ring for aligning the visual indicator of the locking ring corresponding to the tab with the visual indicator of the plunger corresponding to the notch, said alignment between these two visual indicators leading to unlock the locking mechanism. According to one embodiment, the visual indicator is not for marking a dosage indication.

According to one embodiment, the plunger and the locking ring are connected together so that means for blocking the plunger stroke in a locked position, are not visible from the operator. According to one embodiment, the plunger and the locking ring are connected together so that means for unlocking the locked position are not visible from the operator. According to one embodiment, the plunger does not rotate with respect to the piston. Advantageously, this specific configuration forces the operator to think more about the action in progress and in particular the consistency between the compound to be delivered to his patient and that which is in the pre-filled syringe.

According to one specific embodiment, in the lockable syringe of the invention:
the plunger comprises a visual indicator, preferably a colored indicator, on its rod for marking the unlock position of the locking mechanism; and
the locking ring comprises on its outer surface, from four to eight different visual indicators, and has one tab in its inner surface, for blocking the plunger stroke in a locked position.

According to one embodiment, the locking mechanism is made of plastic.

The lockable syringe of the invention is particularly of interest in the medical field. According to one embodiment, the lockable syringe is a pre-filled syringe. Preferably the syringe is pre-filled with a drug, more preferably selected from the following drugs or acceptable salts thereof: midazolam, ropivacaine, lidocaine, mepivacaine, bupivacaine, levobupivacaine, lidocaine epinephrine, naloxone, urapidil, dexamethasone, ondansetron, fentanyl, sufentail, glucose, oxycodone, atracurium, cisatracurium, hydromorphone, alfentanil, prilocaine, chloroprocaine, nicardipine, norepinephrine, propofol, dexmedetomidine, morphine, calcium chloride, calcium gluconate, sodium bicarbonate, furosemide, magnesium sulfate, glycopyrronium, neostigmine, neostigmine/glycopyrronium, rocuronium, sugammadex, ketamine, adenosine, vecuronium, tranexamic acid, amiodarone, diazepam, droperidol, atropine, phenylephrine, ephedrine, suxamethonium, atosiban, baclofen, dobutamine and dopamine.

Another subject of the invention is a method for dispensing a drug to a subject by means of a lockable syringe having a locking mechanism as described above, comprising steps in which:

(a) determining the drug that has to be dispensed to the subject;

(b) identifying the visual indicator corresponding to the drug determined at step (a), among the at least two visual indicators present on the outer surface of the locking ring;

(c) rotating the locking ring so that the visual indicator of the drug identified at step (b) is aligned with a visual indicator on the plunger marking the unlock position of the locking mechanism; and

(d) pushing the plunger so that dispensing the drug pre-filled in the lockable syringe.

According to one embodiment, the method of the invention is not for adjusting a dosage of a drug prefilled in the syringe. Advantageously, the lockable syringe and/or the locking mechanism of the invention may be easily industrialized. Advantageously, the pre-filled syringe of the invention is ergonomic and simple to be used by the operator so that checking step of the drug inside the pre-filled syringe, is fast and easy to implement.

### DEFINITIONS

In the present invention, the following terms have the following meanings:
“Connective rod” refers to an element of the plunger located in its distal part, said element being suitable for solidly fixing the plunger 32 to the piston 5 inside the syringe body 2. Preferably, the connective rod is configured so that, in the locked position of the syringe 1, means 3211 of the distal plate 321 are not aligned with means 312 of the locking ring 31 in order that the rod 322 cannot advance inside the syringe body 2. According to one embodiment, in the unlocked position of the syringe 1, means 3211 of the distal plate 321 are aligned with means 312 of the locking ring 31 in order that the rod 322 can advance inside the syringe body 2.
“Locked position” refers to a configuration of the syringe 1 before the administration to a subject of the drug pre-filled inside said syringe 1, in which the rod 322 cannot advance inside the syringe body 2. Preferably, a locked position is a configuration in which (a) at least 80%, 85%, 90% or 95% of the length of the rod 322 of the plunger 32 is out of the syringe body 2 and of the locking ring 31; and in which (b) means 3211 of the distal plate 321 are not aligned with means 312 of the locking ring 31 so that the rod 322 cannot advance inside the syringe body 2. According to one embodiment, the locked position refers to a configuration of the syringe 1 in which (a) the whole length of the rod 322 of the plunger 32 is out of the syringe body 2 and of the locking ring 31; and in which (b) the configuration of the rod 322 relative to the locking ring 31 is such that the rod 322 cannot advance inside the syringe body 2.
Unlocked position” or “unlock position” refers to a configuration of the syringe 1 in which the rod 322 can advance inside the syringe body 2. Preferably, an “unlocked position” is a configuration in which means 3211 of the distal plate 321 cooperate with means 312 of the locking ring 31 so that the rod 322 can advance inside the syringe body 2.

### DESCRIPTION OF THE DRAWINGS

Features and advantages of the invention will become apparent from the following description of several embodiments of a lockable syringe according to the invention, this description being given merely by way of example and with reference to the appended drawings in which:

FIG. 1 is an exploded perspective view of a lockable syringe of the invention;

FIG. 2 is a longitudinal section of the lockable syringe in a first embodiment of the invention in which the locking mechanism is in a locked position (FIG. 2A) and in a second embodiment in which the locking mechanism is in an unlocked position (FIG. 2B).

FIG. 3 is a perspective view of the adapter comprising a flat surface and a ring;

FIG. 4 is a perspective view of the locking ring comprising a tab and visual indicators;

FIG. 5 is a perspective view of the plunger comprising a rod, a distal plate and a connective rod;

FIGS. 6A and 6B illustrate an enlarged view of the connection between the syringe body and the locking mechanism of FIGS. 2A and 2B, respectively.

### ILLUSTRATIVE EMBODIMENTS OF THE INVENTION

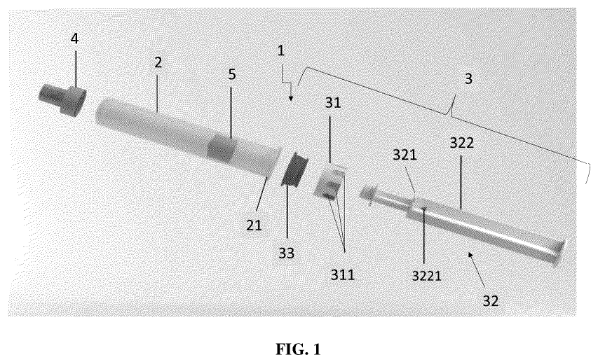

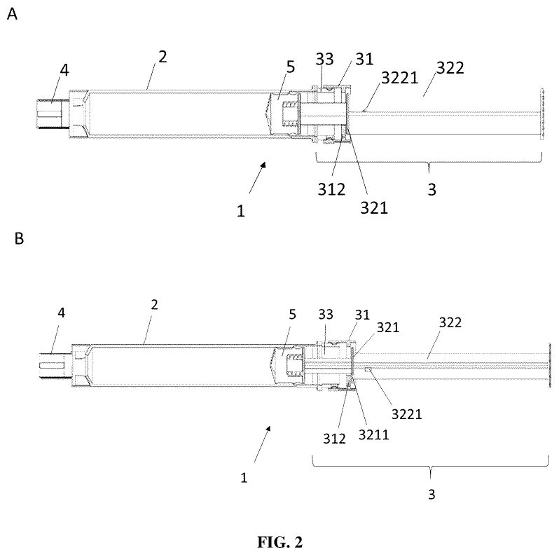

FIG. 1 shows an exploded view of a lockable syringe 1 suitable for safely dispensing of a drug. As visible in FIG. 1, the lockable syringe 1 comprises a syringe body 2 with a tip 4 at its distal end, and a locking mechanism 3 at its proximal end, said locking mechanism 3 being aligned along the longitudinal axis of the syringe body 2 as presented in FIG. 2. Said locking mechanism comprises a locking ring 31, a plunger 32 and an adapter 33.

The locking ring 31, presented in FIG. 4, comprises at least two visual indicators 311 on its outer surface. Especially, the visual indicators 311 are on the proximal outer surface of the locking ring 31. Alternatively, the visual indicators 311 may also be located on the distal outer surface or any other arrangements on the outer surface of the locking ring 31. In FIG. 1 or in FIG. 4, the visual indicators 311 are colored indicators and/or literal indicators and/or numeral indications (such as figures or codes); each of the visual indicators 311 corresponding to one drug or one pharmaceutical composition. According to one embodiment, the visual indicators 311 are each independently selected from colored indicators and literal indicators. According to one embodiment, the drug prefilled in the syringe body 2 is represented on the locking ring 31 by one color and/or literal indicators, preferably the color and the literal indicators representative of the prefilled drug are different from the other visual indicators 311 of the locking ring 31. According to one embodiment, the visual indicators 311 are both colored indicators and literal indicators; and all the visual indicators represent the same color but are different literal indicators. According to one embodiment, the visual indicators 311 are both colored indicators and numeral indicators; and all the visual indicators represent the same color but are different numeral indicators. According to one embodiment, the numeral indicator represents a drug concentration. In the present invention, at least one visual indicator 311 corresponds to the drug or the pharmaceutical composition pre-filled in the syringe body 2. According to one embodiment, only one visual indicator 311 corresponds to the drug or the pharmaceutical composition pre-filled in the syringe body 2. In the present invention, the other visual indicators 311 correspond to other drugs or pharmaceutical compositions than those pre-filled in the syringe body 2, said others drugs or pharmaceutical compositions having the same pharmaceutical function or not, as the pre-filled drug or pharmaceutical composition. According to one embodiment, when the visual indicators 311 are colored indicators, the color is chosen according to the medical classification of drugs according to the norm ISO 26825 2008. Examples of colors used in the medical classification of drugs are presented in the table hereinabove.

| Class of drugs | PMS* color | Examples |
|---|---|---|
| Hypnotics | Primary Yellow C | Thiopental, methohexital, |
| | | propofol, ketamine |
| Benzodiazepine | Orange 151 | Diazepam, midazolam |
| Antagonists of | Orange 151 with white | Flumazenil |
| benzodiazepines | diagonal stripes | |
| Curariants | Fluorescent red 811 or | Suxamethonium, d-tubocurarine, |
| | hot red | pancuronium, atracurium, vecuronium |
| Antagonists of | Fluorescent red 811 or | Neostigmine, edrophonium, |
| curariants | hot red, with white | pyridostigmine |
| | diagonal stripes | |
| Opioids | Blue 297 | Morphine, fentanyl, pethidine |
| Antagonists of opioids | Blue 297 with white | Naloxone |
| | diagonal stripes | |
| Sympathicomimetics | Purple 256 | Adrenalin, ephedrine, |
| | | phenylephrine, metaraminol |
| antihypertensives | Purple 256 with white | Nitroprussiate, nitroglycerin, |
| | diagonal stripes | phentolamine, hydralazine |
| local anesthetics | Grey 401 | Procaine, lidocaine, bupivacaine, |
| | | ropivacaine |
| anticholinergics | Green 367 | Atropine, glycopyrronium |
| antiemetics | Salmon 156 | Droperidol, metoclopramide, |
| | | ondansetron |
| Others drugs | white | Ocytocine, heparin, protamine, |
| | | potassium chloride, tacrine, |
| | | antibiotics, non-steroidal |
| | | anti-inflammatory drugs |

*PMS means Pantone Matching System ® which is the Pantone ® system for comparing colors.

According to one embodiment, the literal indicators 311 may include the name of the drug or the pharmaceutical formulation such as for example, the trademarks or any commercial designation of the medicament, or the abbreviation of the drug or the pharmaceutical formulation such as for example, the trigram of the name of the drug, or the drug concentration.

In the lockable syringe of the invention 1, the locking ring 31 is connected to the syringe body 2 thanks to the adapter 33. According to one embodiment, when connected to the syringe body 2 and/or the adapter 33, the locking ring 31 is able to freely rotate on itself, around the longitudinal axis of the syringe body 2. According to one embodiment, the locking ring 31 is able to freely rotate on itself, around the longitudinal axis of the syringe body 2 without slidably moving along the longitudinal axis of the syringe body 2. According to one embodiment, the locking ring 31 does not slidably move along the longitudinal axis of the syringe body 2. According to one embodiment, when connected to the syringe body 2 and/or the adapter 33, the locking ring 31 does not enclose any part of the syringe body 2. According to one embodiment, the locking ring 31 does not comprise any hinge.

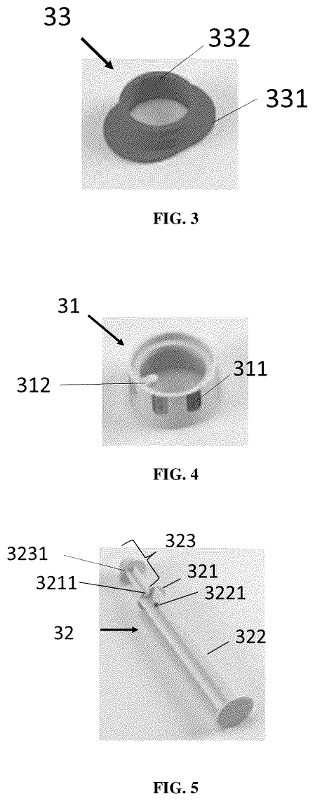

As presented in FIG. 3, the adapter 33 comprises a flat surface 331 surmounted by a ring 332. As presented in FIG. 2, the locking ring 31 is not engaged in the syringe body 2. Thus, the outer surface of the locking ring 31 remains visible from the operator whatever the locked or unlocked configuration of the locking mechanism.

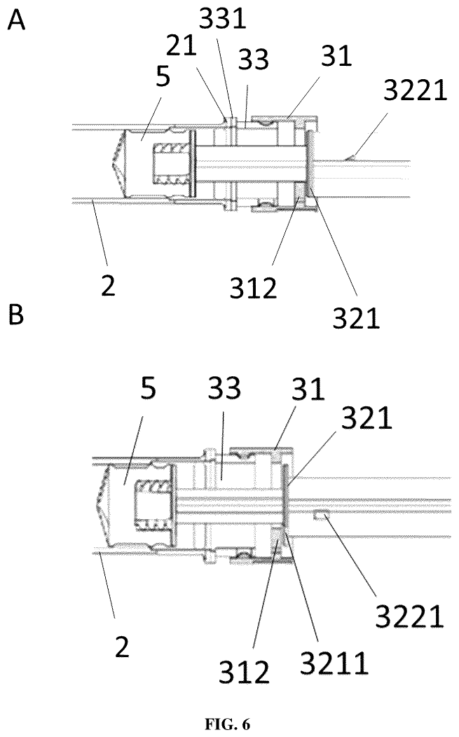

As presented in FIGS. 2 and 6, the flat surface 331 of the adapter 33 is clipped on the flanges 21 of the syringe body 2 at its proximal end so that the adapter 33 is securely attached without impeding the grip of the syringe by the operator at the level of the flanges. The ring 332 of the adapter 33 has means for connecting the locking ring 31. In particular, when the adapter 33 and the locking ring 31 are attached to each other, the ring 332 of the adapter 33 disappears in the partially or totally inner volume of the locking ring 31. According to one embodiment, the locking ring 31 connects to the ring 332 of the adapter 33 thanks to a projection in its inner surface, said projection being configured for fitting with the at least one groove (preferably, an outer peripherical groove) present inside the outer surface of the ring 332 of the adapter 33.

The locking ring 31 further comprises means 312 for blocking the plunger stroke in a locked position. According to one embodiment, means 312 for blocking the plunger stroke include at least one tab in the inner volume of the locking ring 31, as presented in FIGS. 2 and 4. Especially, the longitudinal axis of the tab as presented in FIG. 4, is transvers to the longitudinal axis of the locking ring 31 and/or of the longitudinal axis of the syringe body 2. According to one embodiment, the tab is located on an inner diameter of the locking ring 31 and at the proximal end of said locking ring 31.

The plunger 32, presented in FIG. 5, comprises at least one distal plate 321, a rod 322 and a connective rod 323. As seen in FIG. 5, the distal plate 321 is located between the rod 322 and the connective rod 323. According to one embodiment, the connective rod 323 has a length higher than the length of the locking ring and lower than the length of the rod 322. According to one embodiment, the connective rod 323 has a diameter lower than the diameter of the rod 322. The distal plate 321 includes means 3211 for cooperating with means 312 of the locking ring 31 so that it is possible to either prevent (locking position) or enable (unlocking position) the advancement of the plunger, preferably of the rod 322, inside the syringe body 2. The rod 322 comprises a finger grip. According to one embodiment, the rod 322 comprises at least 2 wings, preferably 3, 4 or 5 wings. According to one embodiment, the rod 322 does not comprise any wing. According to one embodiment, the rod 322 is cylindrical. According to one embodiment, the cross section of the rod 322 is a cross. According to one embodiment, the rod 322, preferably the wings of the rod 322, does not comprise any rib portions.

According to one embodiment, the plunger 32 and/or the rod 322 does not comprise any grooves. According to one embodiment, the plunger 32 and/or the rod 322 does not comprise any grooves of variable length. According to one embodiment, the plunger 32 and/or the rod 322 does not comprise any grooves of variable length wherein the length of the grooves corresponds to a dosage indicator. According to one embodiment, the plunger 32 and/or the rod 322 does not comprise means for partially advancing inside the syringe body 2. According to one embodiment, the plunger 32 does not rotate on itself or around the longitudinal axis of the syringe body 2. According to one embodiment, the plunger 32 does not rotate with respect to the piston 5. Especially, as presented in FIGS. 1 and 2, the rod 322 includes visual means 3221 for marking an unlock position; said visual means 3221 being visible by the operator when the syringe mechanism is locked.

According to one embodiment, visual means 3221 may be colored indicators, literal indicators and/or any pictograms. According to one embodiment, said visual means 3221 for marking an unlock position do not include means for unlocking the locking system. According to one embodiment, said visual means 3221 are not dosage indicators. According to one embodiment, said visual means 3221 are not for adjusting the dose of a drug. In an alternative embodiment, the rod 322 includes tangible means for marking an unlock position such as for example a serigraphy, a projection, a groove, or any other suitable means.

As clearly visible in FIG. 5, visual means 3221 on the rod 322 points out a position on the distal plate 321 of a specific arrangement able to cooperate with means 312 for unlocking the locking mechanism 3 and allowing freely moving the plunger 32 inside the syringe body 2. The plunger 32 also comprises a connective rod 323 for solidly fixing the plunger 32 to the piston 5 inside the syringe body 2. The connective rod 323 comprises at its distal end, a plate 3231, preferably having the same diameter as that of the distal plate 321.

According to one embodiment, the syringe body 2, the piston 5, the adapter 33, the locking ring 31 and/or the plunger 32 is (are) made of plastic. According to one embodiment, the piston 5 may be made of an elastomer or a silicone.

A method of dispensing a material (preferably a drug or a pharmaceutical composition) by means of the lockable syringe 1 according to the invention, comprises steps as described below.

Initially, the lockable pre-filled syringe 1 is in the storage configuration shown in FIGS. 2A and 6A, in which the rod 322 of the plunger 32 is out of the syringe body 2 and of the locking ring 31.

The lockable syringe has been previously filled with a component selected from any drugs or pharmaceutical compositions in a form suitable for parenteral administration (such as by intravenous, intramuscular or subcutaneous injection or intravenous infusion).

In this configuration, the locking mechanism 3 is in a locked position: the notch 3211 present on the distal plate 321, is not aligned with the tab 312 of the locking ring 31 so that the plunger stroke inside the syringe body 2 is blocked. As seen in FIGS. 2A and 6A, the tab 312 is a stopper for the distal plate 321. The tab 312 is preferably located in the inner volume of the locking ring 31. Consequently, the drug pre-filled in the syringe cannot be dispensed.

For unlocking the locking mechanism 3, the operator has to first identify the component pre-filled inside the syringe 1 and then, to locate the visual indicator 311 corresponding to this pre-filled component, on the locking ring 31. According to one embodiment, the visual indicator 311 is on the outer surface of the locking ring 31.

Secondly, the operator has to rotate the locking ring 31 on itself about the longitudinal axis of the syringe body 2 in order to fit the visual indicator 311 corresponding to this pre-filled component on the locking ring 31, with the visual indicator 3221 of the rod 322 of the plunger 32. In this way, the tab 312 of the locking ring 31 is aligned with the notch 3211 of the plunger 32 (as seen FIGS. 2B and 6B), leading to free the stroke of the plunger inside the locking ring 31 and of the syringe body 2.

Finally, the operator has to push the plunger inside the syringe body for dispensing the component pre-filled inside said syringe.

Advantageously, the locking mechanism 3 of the invention makes the operator more aware of the use of the pre-filled syringe, and in particular of the content of the syringe that the operator is preparing to deliver. Indeed, for using the lockable syringe of the invention, the operator has to be sure of its content, and the lockable mechanism will be unlocked only if the content has been properly identified by the operator. Consequently, the lockable syringe of the invention is safer than conventional pre-filled syringes.

The invention is not limited to the examples described and shown. In particular, other materials and shapes than those described above can be considered for the constitutive members of a lockable syringe according to the invention.

1-15. (canceled)

16. A lockable syringe for dispensing a drug, comprising:
a syringe body having a longitudinal axis and comprising a distal end and a proximal end; and
a locking mechanism;
wherein:
said locking mechanism is configured to be attached to the proximal end of the syringe body, and said locking mechanism comprises:
a plunger having at least one distal plate, and a rod with visual means for marking an unlock position; and
a locking ring which is free in its rotation about the longitudinal axis of the syringe body said locking ring has at least one visual indicator on its outer surface, and said locking ring has means for blocking the plunger stroke in a locked position.

17. The lockable syringe according to claim 16, wherein the locking mechanism further comprises an adapter configured to be attached to the proximal end of the syringe body and comprising means for receiving the locking ring.

18. The lockable syringe according to claim 17, wherein the adapter is molded with the syringe body.

19. The lockable syringe according to claim 16, wherein means for receiving the locking ring comprise at least one groove.

20. The lockable syringe according to claim 17, wherein the adapter is located on the flanges of the syringe body.

21. The lockable syringe according to claim 16, wherein the locking ring comprises at least two visual indicators which may be of the same or different nature.

22. The lockable syringe according to claim 16, wherein means for blocking the plunger stroke in a lock position comprise at least one tab.

23. The lockable syringe according to claim 16, wherein when means for blocking the plunger stroke in a lock position comprise at least one tab, the plunger comprises at least one notch on the distal plate configured for receiving said tab and providing an unlock position.

24. The lockable syringe according to claim 23, wherein the notch is marked by a visual indicator on the rod of the plunger.

25. The lockable syringe according to claim 16, wherein the plunger and the locking ring are connected together so that means for blocking the plunger stroke in a locked position, are not visible from the operator.

26. The lockable syringe according to claim 22, wherein the at least one tab is aligned on one visual indicator present on the outer surface of the locking ring.

27. The lockable syringe according to claim 16, wherein the locking mechanism is made of plastic.

28. The lockable syringe according to claim 16, wherein the lockable syringe is a pre-filled syringe.

29. The lockable syringe according to claim 28, wherein the syringe is pre-filled with a drug.

30. The lockable syringe according to claim 29, wherein the drug is selected from the following drugs or acceptable salts thereof: midazolam, ropivacaine, lidocaine, mepivacaine, bupivacaine, levobupivacaine, lidocaine epinephrine, naloxone, urapidil, dexamethasone, ondansetron, fentanyl, sufentail, glucose, oxycodone, atracurium, cisatracurium, hydromorphone, alfentanil, prilocaine, chloroprocaine, nicardipine, norepinephrine, propofol, dexmedetomidine, morphine, calcium chloride, calcium gluconate, sodium bicarbonate, furosemide, magnesium sulfate, glycopyrronium, neostigmine, neostigmine/glycopyrronium, rocuronium, sugammadex, ketamine, adenosine, vecuronium, tranexamic acid, amiodarone, diazepam, droperidol, atropine, phenylephrine, ephedrine, suxamethonium, atosiban, baclofen, dobutamine and dopamine.

31. The lockable syringe according to claim 16, wherein:
the plunger comprises a colored indicator on its rod, for marking the unlock position of the locking mechanism; and
the locking ring comprises on its outer surface, from 4 to 8 different visual indicators, and has one tag in its inner surface, for blocking the plunger stroke in a locked position.
